(19) United States
(12) Patent Application Publication
(10) Pub. No.: US 2023/0233734 A1
(43) Pub. Date: Jul. 27, 2023

(54) AIR STERILIZATION AND DISINFECTION MODULE WITH TWO BACTERICIDAL FACTORS

(71) Applicant: SHENZHEN YITOA INTELLIGENT INDUSTRIAL CO., LTD, Shenzhen City (CN)

(72) Inventors: JUNHUANG ZHUANG, SHENZHEN CITY (CN); ZHIHONG ZHENG, SHENZHEN CITY (CN)

(21) Appl. No.: 17/717,048

(22) Filed: Apr. 9, 2022

(30) Foreign Application Priority Data
Jan. 25, 2022 (CN) 202220210212.2

(51) Int. Cl. A61L 9/20

(52) U.S. Cl. CPC A61L 9/205; A61L 2209/16

(57) ABSTRACT

The present disclosure is related to an air sterilization-and-disinfection module that embodies two bactericidal factors. The module is directed to a field of sterilization for an air conditioning conduit. A corrugated board is disposed in the middle of the module. A plurality of wavy channels are formed in between multiple corrugated boards. Several metal meshes are disposed at top and bottom of the multiple corrugated boards. A sterilization component is disposed inside the wavy channels for sterilizing the air conditioning conduits. The two bactericidal factors are pure ultraviolet irradiation sterilization and photocatalyst sterilization. The sterilization component irradiates ultraviolet ray and the ultraviolet ray is spilled out from top and bottom of the wavy channels since the light is reflected by a reflective layer on the surfaces of wavy channels. The spilled ultraviolet ray stimulates the photocatalyst mesh on the metal meshes for achieving the two bactericidal factors.

### CROSS-REFERENCE TO RELATED PATENT APPLICATION

This application claims the benefit of Priority to Patent Application No. 202220210212.2, filed on Jan. 25, 2022 in People’s Republic of China. The entire content of the above identified application is incorporated herein by reference.

Some references, which may include patents, patent applications and various publications, may be cited and discussed in the description of this disclosure. The citation and/or discussion of such references is provided merely to clarify the description of the present disclosure and is not an admission that any such reference is “prior art” to the disclosure described herein. All references cited and discussed in this specification are incorporated herein by reference in their entireties and to the same extent as if each reference was individually incorporated by reference.

### FIELD OF THE DISCLOSURE

The present disclosure is directed to a technical field of air conditioning conduit sterilization, and more particularly to an air sterilization-and-disinfection module that embodies two bactericidal factors.

### BACKGROUND OF THE DISCLOSURE

Air conditioners are widespread use in various occasions such as super markets, shopping malls, hotels, schools, offices and buildings in our daily life. For The ventilation principle of a central-controlled air conditioner is to utilize a ventilation conduit to deliver air flow and to modulate some parameters such as temperature, humidity, cleanness and current velocity in a closed space for meeting requirements of human comfort and technologies. The main functions of an air conditioner include heating, cooling and ventilation.

The conduit of a conventional air conditioner can be equipped with an air sterilization device that conducts sterilization when the air conditioner is in operation. The conduit can be sterilized by irradiating the air flow with ultraviolet ray. The ultraviolet ray can be used to catalyze photocatalyst via an ultraviolet lamp or an LED light board. However, the conventional device lacks a structure to embody the two bactericidal factors at the same time. For example, the photocatalyst can be used to achieve one of the bactericidal factors, but it is difficult to design a structure that can conduct sterilization with the photocatalyst and also use the ultraviolet ray to sterilize the air at the same time.

### SUMMARY OF THE DISCLOSURE

The invention of the present disclosure is to solve the shortcomings of the conventional technologies, an air sterilization-and-disinfection module that embodies two bactericidal factors is provided. A sterilization component is provided in the air sterilization-and-disinfection module for irradiating an ultraviolet ray. The ultraviolet ray is spilled out for stimulating the photocatalyst mesh formed on the metal meshes for achieving the two bactericidal factors, i.e., pure ultraviolet irradiation sterilization and photocatalyst sterilization. In the meantime, the honeycomb-shaped photocatalytic mesh has lower air resistance and will not be affected by the passing air. The honeycomb-shaped photocatalytic mesh has a larger area than other shapes of mesh, and the larger area attached with the photocatalyst provides better effect.

For implementing the above-mentioned subjective, the air sterilization-and-disinfection module that embodies two bactericidal factors can be adapted to an air conditioning conduit. The air sterilization-and-disinfection module includes multiple corrugated boards that are disposed in the middle inside the main body of the air sterilization-and-disinfection module, multiple wavy channels formed inside the corrugated boards, at least two metal meshes disposed at top and bottom of the corrugated boards, a photocatalytic mesh disposed on a surface of the metal mesh, and a sterilization component disposed inside the wavy channels for sterilizing the air conditioning conduit.

Further, the sterilization component includes a plurality of UVC-LED light boards disposed inside the wavy channels, a reflective layer formed on the surface of each of the wavy channels and outside the UVC-LED light boards, and a photocatalyst mesh formed on the metal meshes.

Still further, a groove is formed in the middle outside the corrugated board.

Further, if the spaces between the multiple corrugated boards are the same, the widths of the wavy channels are larger than or equal to one centimeter.

Still further, the photocatalytic mesh is a Titanium dioxide photocatalyst.

Further, the photocatalytic mesh can be a honeycomb-shaped structure.

The present disclosure provides an air sterilization-and-disinfection module that embodies two bactericidal factors. The air sterilization-and-disinfection module provides following beneficial effects.

According to one of the beneficial effects of the present disclosure, a sterilization component is provided in the air sterilization-and-disinfection module. The UVC-LED light boards are provided to emit ultraviolet rays inside the wavy channels so as to irradiate the air flow passing the wavy channels with the ultraviolet rays. The ultraviolet ray is reflected by a reflective layer on the surfaces of the wavy channels and spilled from top and bottom of the wavy channels. The spilled ultraviolet ray just enough stimulates the photocatalyst mesh on the metal meshes and then achieves the two bactericidal factors, i.e., pure ultraviolet irradiation sterilization and photocatalyst sterilization.

According to one further beneficial effect of the present disclosure, the honeycomb-shaped photocatalytic mesh has lower air resistance and will not be affected by the passing air. It should be noted that the shape of photocatalytic mesh can be, but not limited to, honeycomb, square, rectangle or diamond hole. The honeycomb-shaped photocatalytic mesh has a larger area than other shapes of mesh, and the larger area attached with the photocatalyst provides better effect.

These and other aspects of the present disclosure will become apparent from the following description of the embodiment taken in conjunction with the following drawings and their captions, although variations and modifications therein may be affected without departing from the spirit and scope of the novel concepts of the disclosure.

### BRIEF DESCRIPTION OF THE DRAWINGS

The described embodiments may be better understood by reference to the following description and the accompanying drawings, in which:

FIG. 1 is a schematic diagram depicting an overall structure of an air sterilization-and-disinfection module according to one embodiment of the present disclosure;

FIG. 2 is a schematic diagram depicting a structure of a corrugated board according to one embodiment of the present disclosure;

FIG. 3 is a schematic diagram depicting a magnified view of portion “A” of FIG. 1 in one embodiment of the present disclosure;

FIG. 4 is a schematic diagram depicting a top view of the corrugated board according to one embodiment of the present disclosure; and

FIG. 5 is a schematic diagram depicting a sectional view of a main body of the air sterilization-and-disinfection module according to one embodiment of the present disclosure.

### DETAILED DESCRIPTION OF THE EXEMPLARY EMBODIMENTS

The present disclosure is more particularly described in the following examples that are intended as illustrative only since numerous modifications and variations therein will be apparent to those skilled in the art. Like numbers in the drawings indicate like components throughout the views. As used in the description herein and throughout the claims that follow, unless the context clearly dictates otherwise, the meaning of “a”, “an”, and “the” includes plural reference, and the meaning of “in” includes “in” and “on”. Titles or subtitles can be used herein for the convenience of a reader, which shall have no influence on the scope of the present disclosure.

The terms used herein generally have their ordinary meanings in the art. In the case of conflict, the present document, including any definitions given herein, will prevail. The same thing can be expressed in more than one way. Alternative language and synonyms can be used for any term(s) discussed herein, and no special significance is to be placed upon whether a term is elaborated or discussed herein. A recital of one or more synonyms does not exclude the use of other synonyms. The use of examples anywhere in this specification including examples of any terms is illustrative only, and in no way limits the scope and meaning of the present disclosure or of any exemplified term. Likewise, the present disclosure is not limited to various embodiments given herein. Numbering terms such as “first”, “second” or “third” can be used to describe various components, signals or the like, which are for distinguishing one component/signal from another one only, and are not intended to, nor should be construed to impose any substantive limitations on the components, signals or the like.

The present disclosure provides an air sterilization-and-disinfection module that embodies two bactericidal factors. The air sterilization-and-disinfection module has a sterilization component that can use spilled ultraviolet ray to stimulate photocatalytic meshes disposed on the metal meshes. The bactericidal factors, i.e., pure ultraviolet irradiation sterilization and photocatalyst sterilization, can be implemented. In particular, the photocatalytic mesh can be designed in a honeycomb-shaped structure that has a lower air resistance and will not be affected by a passing air. The honeycomb-shaped structure has a larger area than other types of meshes, and the larger area attached with the photocatalyst provides better effect.

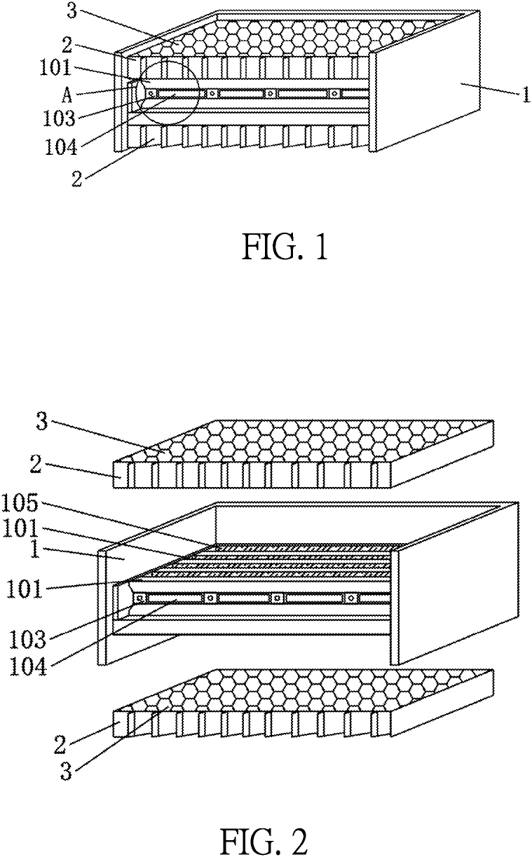

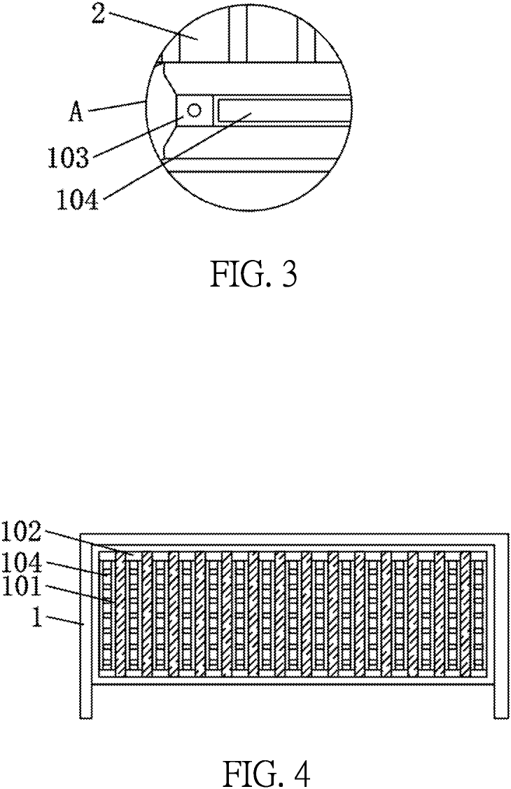

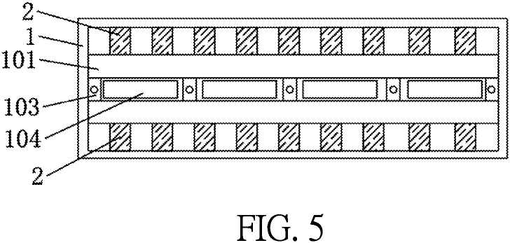

References are made to FIGS. 1-5. An air sterilization-and-disinfection module that embodies two bactericidal factors is provided. The air sterilization-and-disinfection module can be adapted to an air conditioning conduit. The air sterilization-and-disinfection module includes multiple corrugated boards 101 disposed in the middle and inside a module body 1, multiple wavy channels 102 disposed in between the multiple corrugated boards 101, a plurality of grooves 103 and UVC-LED light boards 104 mounted in the module via the multiple wavy channels 102, at least two metal meshes disposed at both top and bottom of the corrugated boards 101, one or more photocatalytic meshes 3 on the surfaces of the metal meshes 2, and a sterilization component that is disposed inside the wavy channels 102 for sterilizing the air conditioning conduit.

Further, the sterilization component includes a plurality of UVC-LED light boards 104 disposed inside the wavy channels 102, a reflective layer 105 formed on inner surfaces of the wavy channels 102 and outside the UVC-LED light boards 104, and one or more photocatalytic meshes 3 on the surfaces of the metal meshes 2. Through arrangements of the wavy channels 102, the UVC-LED light boards 104, the reflective layer 105, the phptocatalytic meshes 3, and the module body 1 being installed inside an air sterilization-and-disinfection device or an air conditioning conduit, the UVC-LED light boards 104 emits ultraviolet rays inside the wavy channels 102 for sterilizing the air flow passing the wavy channels 102. Further, the reflective layer 105 reflects the ultraviolet rays emitted to the wavy channels 102 and forms the spilled ultraviolet rays that can be used to stimulate the photocatalytic meshes 3 disposed on the metal meshes 2. Accordingly, the two bactericidal factors including pure ultraviolet irradiation sterilization and photocatalyst sterilization can be achieved.

Still further, several grooves 103 are formed in the middle and outside the corrugated boards 101. The arrangement of the grooves 103 allows the UVC-LED light boards 104 to be disposed outside the corrugated boards 101, and therefore the UVC-LED light boards 104 can be prevented from falling out when the module is in use.

Further, the spaces between the corrugated boards 101 are the same, and the width of each of the wavy channels 102 that are formed between the corrugated boards 101 is larger than or equal to one centimeter. The wavy channels 102 make the ultraviolet rays irradiating the passing air for sterilization.

Still further, the photocatalytic mesh 3 can be Titanium dioxide photocatalyst. The Titanium dioxide is used as the photocatalytic material that can be used to decompose the organic compounds, parts of inorganic compounds, bacteria and viruses because the active species such as strong oxidizing hydroxyl radicals and superoxide radicals are produced when the ultraviolet ray irradiates the Titanium dioxide.

Further, the photocatalytic mesh 3 can be designed as a honeycomb-shaped structure. The honeycomb-shaped photocatalytic mesh 3 has a lower air resistance and will not be affected by the passing air. The shape of the photocatalytic mesh 3 can be, but not limited to, honeycomb, square, rectangle or diamond hole. In particular, the honeycomb-shaped photocatalytic mesh has a larger area than other shapes of mesh, and the larger area attached with the photocatalyst provides better effect.

In the above embodiments, the description with respect to each of the embodiments has its own emphasis, and parts of some of the embodiments that lack details can refer to the descriptions of other embodiments.

The foregoing description of the exemplary embodiments of the disclosure has been presented only for the purposes of illustration and description and is not intended to be exhaustive or to limit the disclosure to the precise forms disclosed. Many modifications and variations are possible in light of the above teaching.

The embodiments were chosen and described in order to explain the principles of the disclosure and their practical application so as to enable others skilled in the art to utilize the disclosure and various embodiments and with various modifications as are suited to the particular use contemplated. Alternative embodiments will become apparent to those skilled in the art to which the present disclosure pertains without departing from its spirit and scope.

What is claimed is:

1. An air sterilization-and-disinfection module that embodies two bactericidal factors, adapted to an air conditioning conduit, comprising:
multiple corrugated boards (101) disposed in the middle inside the air sterilization-and-disinfection module;
multiple wavy channels (102) disposed in between the multiple corrugated boards (101);
at least two metal meshes (2) disposed at top and bottom of the multiple corrugated boards (101);
a photocatalytic mesh (3) formed on each of the metal meshes (2); and
a sterilization component disposed inside the multiple wavy channels (102) for sterilizing the air conditioning conduit.

2. The air sterilization-and-disinfection module according to claim 1, wherein the sterilization component comprises:
a plurality of UVC-LED light boards (104) disposed inside the multiple wavy channels (102); and
a plurality of reflective layers (105) formed on the surfaces of the wavy channels (102) and outside the plurality of UVC-LED light boards (104).

3. The air sterilization-and-disinfection module according to claim 1, further comprising multiple grooves disposed in the middle outside the multiple corrugated boards (101).

4. The air sterilization-and-disinfection module according to claim 1, wherein the spaces between the corrugated boards (101) are the same, and the width of each of the wavy channels (102) is larger than or equal to one centimeter.

5. The air sterilization-and-disinfection module according to claim 1, wherein photocatalytic mesh (3) is a Titanium dioxide photocatalyst.

6. The air sterilization-and-disinfection module according to claim 1, wherein the photocatalytic mesh (3) is a honeycomb-shaped structure.
